(19) United States
(12) Patent Application Publication
(10) Pub. No.: US 2026/0069647 A1
(43) Pub. Date: Mar. 12, 2026

(54) TRADITIONAL CHINESE MEDICINE COMPOUND COMPOSITION FOR TREATING HEART FAILURE AND YANG-WARMING AND HEART-SETTLING PLASTER

(71) Applicant: The First Affiliated Hospital of Anhui University of Traditional Chinese Medicine (Ahnui, Hefei (CN)

(72) Inventors: Lan Ge, Hefei (CN); Xiaoyu Cheng, Hefei (CN); Zhenpeng Zhu, Hefei (CN); Zhongzhi Li, Hefei (CN); Wenjun Jiang, Hefei (CN); Dan Cheng, Hefei (CN); Yulong Liu, Hefei (CN); Xinyue Wang, Hefei (CN); Maomao Zhang, Hefei (CN); Xiaowen Ma, Hefei (CN); Mengzhao Wei, Hefei (CN); Sheng Sheng, Hefei (CN); Mengru Chen, Hefei (CN)

(21) Appl. No.: 19/318,330

(22) Filed: Sep. 3, 2025

(30) Foreign Application Priority Data
Sep. 6, 2024 (CN) 202411245099.1

(51) Int. Cl. A61K 36/31; A61K 9/00; A61K 35/64; A61K 36/47; A61K 36/54; A61K 36/65; A61K 36/714; A61K 36/90; A61P 9/04

(52) U.S. Cl. CPC A61K 36/31; A61K 9/0014; A61K 35/64; A61K 36/47; A61K 36/54; A61K 36/65; A61K 36/714; A61K 36/90; A61P 9/04

(57) ABSTRACT

A traditional Chinese medicine (TCM) compound composition for treating heart failure (HF) includes Semen Sinapis Albae, Radix Kansui Praeparata, Semen Descurainiae, Ramulus Cinnamomi, Radix Aconiti Lateralis Praeparata, Eupolyphaga Steleophaga, Cortex Moutan, and Rhizoma Smilacis Glabrae. An acupoint selection principle for the external application of a YANG-warming and heart-settling plaster focuses on warming and tonifying the heart and kidneys, promoting blood circulation, and dispelling water retention. Five acupoints are selected: Danzhong (CV17), Xinshu (BL15), Juque (CV14), Shenshu (BL23), and Jingmen (GB25). Among these, Xinshu (BL15) paired with Juque (CV14), and Shenshu (BL23) paired with Jingmen (GB25), form combinations for the heart and kidneys for the heart and kidneys, facilitating tonification of the heart and kidneys, promoting water drainage to resolve dampness, and activating blood circulation to unblock meridians.

### CROSS-REFERENCE TO RELATED APPLICATION

This application claims priority of Chinese Patent Application No. 202411245099.1, filed on Sep. 6, 2024, the entire contents of which are incorporated herein by reference.

### TECHNICAL FIELD

The present disclosure relates to the technical field of traditional Chinese medicine (TCM) pharmaceuticals, and specifically to a TCM compound composition for treating heart failure (HF) and a YANG-warming and heart-settling plaster.

### BACKGROUND

HF is a clinical syndrome characterized by impaired ventricular filling and/or ejection capacity resulting from structural and/or functional abnormalities of the heart. The global prevalence of HF among adults ranges from 1% to 3%, while the age-standardized prevalence rate in China is 1.1%. HF predominantly affects older adults, with patients commonly presenting with three or more comorbid diseases. Chronic heart failure (CHF) exhibits significant heterogeneity, with complex and diverse etiologies and pathophysiological alterations. Disease progression frequently involves multi-organ damage affecting cardiac, renal, hepatic, and gastrointestinal systems. Consequently, numerous Western medications become contraindicated due to organ impairment or hypotension-related complications.

Professor Cheng Xiaoyu, the 10th-generation inheritor of the Cheng's Internal Medicine lineage (Northern She County, Xin'an tradition) and a provincial-level eminent TCM practitioner of Anhui Province, has prescribed a YANG-warming and heart-settling plaster for external acupoint application based on the distinctive features of Xin'an medicine and long-term clinical observation, significantly alleviating clinical symptoms in HF patients, reducing atoxic side effects, thereby improving the quality of life.

### SUMMARY

To solve the technical problems, the present disclosure provides a TCM compound composition for treating HF and a YANG-warming and heart-settling plaster. Prepared according to the monarch-minister-assistant-courier principle of formulation, this compound composition adopts pattern-based differentiation and treatment by combining heart-kidney warming and tonifying herbs with dampness-resolving diuretics and blood-activating meridian-dredging herbs. It targets the root deficiency (Ben) underlying HF while addressing symptomatic manifestations (Biao) through blood activation, meridian unblocking, diuresis, and dampness resolution, thereby achieving dual Biao-Ben treatment and establishing reliable clinical efficacy.

To realize the above objective, the present disclosure employs the following technical solutions.

A TCM compound composition for treating HF, is prepared from the following herbal raw materials in parts by weight:

1-3 parts of Semen Sinapis Albae, 1-3 parts of Radix Kansui Praeparata, 3-5 parts of Semen Descurainiae, 3-5 parts of Ramulus Cinnamomi, 2-4 parts of Radix Aconiti Lateralis Praeparata, 4-6 parts of Eupolyphaga Steleophaga, 4-6 parts of Cortex Moutan, and 2-4 parts of Rhizoma Smilacis Glabrae.

Herbal actions and compatibility rationale for each component in this formulation are as follows.

### Semen Sinapis Albae

[Properties and channel tropism] It has acrid and warm properties, with channel tropism directed toward the lung and stomach meridians.

[Actions] It dispels QI stagnation and eliminates phlegm obstruction, warms the middle jiao to dissipate cold, and unblocks collaterals to alleviate pain.

[Applications] It treats cough and asthma due to phlegm-fluid retention, distension and fullness pain in the chest and hypochondrium, nausea and vomiting, aphasia due to stroke, numbness and pain in the limbs, beriberi, YIN-type flat abscess, swelling toxin, traumatic swelling and pain.

[Modern pharmacology] It exhibits anti-inflammatory and antiemetic effects.

### Radix Kansui Praeparata

[Properties and channel tropism] It has bitter, cold, and toxic properties, with channel tropism directed toward the lung, kidney, and large intestine meridians.

[Actions] It expels water and drains fluid retention.

[Applications] It treats edema and abdominal distension, fluid retention, thoracic congestion, epilepsy, dysphagia, abdominal masses and accumulations, constipation, and dysuria.

[Modern Pharmacology] It exhibits laxative and diuretic effects.

### Semen Descurainiae

[Properties and channel tropism] It has acrid-bitter and cold properties, with channel tropism directed toward the lung, heart, spleen, and bladder meridians.

[Actions] It primarily descends QI and promotes water drainage.

[Applications] It treats severe dyspnea due to lung congestion, phlegm-retention cough, and edema with distension.

[Modern pharmacology] It exhibits cardiotonic and diuretic effects.

### Ramulus Cinnamomi

[Properties and channel tropism] It has acrid-sweet and warm properties, with channel tropism directed toward the bladder, heart, and lung meridians.

[Actions] It induces diaphoresis, releases the muscle layer, warms meridians, and unblocks vessels.

[Applications] It treats wind-cold exterior syndrome, soreness and pain in the shoulders/back/limbs, chest impediment with phlegm retention, amenorrhea, and abdominal masses.

[Modern pharmacology] It exhibits antimicrobial, antiviral, and diuretic activities.

### Radix Aconiti Lateralis Praeparata

[Properties and channel tropism] It has acrid-sweet, hot, and toxic properties, with channel tropism directed toward the heart, spleen, and kidney meridians.

[Actions] It restores YANG to rescue collapse, tonifies fire to support YANG, disperses cold, and eliminates dampness.

[Applications] It treats YIN excess repelling YANG, YANG collapse with profuse sweating, vomiting/diarrhea with reversal-cold limbs, cold-pain in the epigastrium/abdomen, spleen deficiency with cold-dysentery, edema due to foot QI disease, chronic infantile convulsions, Bi syndrome from wind-cold-dampness, traumatic contracture and spasm, impotence from YANG deficiency, uterine coldness, YIN-type ulcerations with discharge, and all disorders from entrenched cold pathogens.

[Modern pharmacology] It exhibits anti-inflammatory effects, glucocorticoid-mimetic activity, analgesic action, sedative properties, positive inotropic effect, hypertensive action, positive chronotropic effect, antiarrhythmic activity, peripheral vasodilating effect, myocardial perfusion-enhancing effect, procoagulant function, hypoglycemic effect, and immunopotentiating activity.

### Eupolyphaga Steleophaga

[Properties and channel tropism] It has salty, cold, and mildly toxic properties, with channel tropism directed toward the liver channel.

[Actions] It breaks blood stasis and reunites tendons/bones.

[Applications] It is used for traumatic tendon/bone fractures, amenorrhea due to blood stasis, and abdominal masses/accumulations.

[Modern pharmacology] It exhibits anticoagulant and lipid-modulating effects.

### Cortex Moutan

[Properties and channel tropism] It has bitter, acrid, and slightly cold properties, with channel tropism directed toward the heart, liver, and kidney meridians.

[Actions] It clears heat and cools blood, and activates blood circulation to resolve stasis.

[Applications] It is used for maculae induced by warm toxins, hematemesis and epistaxis, night fever abating at dawn with tidal fever, amenorrhea and dysmenorrhea, sores and toxin-induced swellings, and traumatic pain.

[Modern pharmacology] It exhibits myocardial ischemia-alleviating effects, antiarrhythmic activity, hypotensive action, anti-atherosclerotic properties, antipyretic effects, sedative properties, anti-inflammatory activity, antibacterial action, anticoagulant effects, immunomodulatory activity, lipid-modulating effects, antitumor activity, anti-implantation effects, and diuretic action.

### Rhizoma Smilacis Glabrae

[Properties and channel tropism] It has sweet-bland and mild properties, with channel tropism directed toward the liver and stomach meridians.

[Actions] It resolves toxins, eliminates dampness, and facilitates joint movement.

[Applications] It treats syphilis, turbid strangury, muscle/bone contracture pain, foot QI with edema, malignant boils, abscesses, and scrofula.

[Modern pharmacology] It exhibits antitumor and detoxifying effects.

The YANG-warming and heart-settling plaster is applied externally at the following acupoints: Danzhong (CV17), Xinshu (BL15), Juque (CV14), Shenshu (BL23), and Jingmen (GB25). Detailed locations are as follows.

### Danzhong (CV17)

[Location] On the anterior median line of the chest, at the level of the fourth intercostal space, midpoint of the line connecting the two nipples.

[Indications] Cough, dyspnea, expectoration of purulent blood, Chest impediment with heart pain, palpitations, irritability, postpartum insufficient lactation, dysphagia with hiccups, and abdominal distension.

### Xinshu (BL15)

[Location] On the back, inferior to the spinous process of the fifth thoracic vertebra (T5), 1.5 cun lateral to the posterior median line.

[Indications] Cardiac pain, palpitations with fright, cough, hematemesis, insomnia, forgetfulness, night sweating, dream-related seminal emission, and epilepsy.

### Juque (CV14)

[Location] In the upper abdomen, on the anterior median line, 6 cun superior to the umbilicus.

[Indications] Chest pain, cardiac pain, irritability and palpitations with fright, corpse syncope, mania and psychosis, epileptic disorders, forgetfulness, chest fullness with shortness of breath, cough with QI counterflow, abdominal distension with acute pain, vomiting and hiccups, dysphagia and regurgitation, acid regurgitation, jaundice, and diarrhea.

### Shenshu (BL23)

[Location] In the lumbar region, inferior to the spinous process of the second lumbar vertebra (L2), 1.5 cun lateral to the posterior median line.

[Indications] Dizziness, tinnitus, deafness, chronic diarrhea, asthma, lumbar soreness and pain, seminal emission, impotence, male infertility, enuresis, urinary retention, irregular menstruation, leukorrhea, female infertility, and wasting-thirst disorder.

### Jingmen (GB25)

[Location] In the lateral lumbar region, approximately 1.8 cun posterior to Zhangmen (LR13), inferior to the free end of the 12th rib.

[Indications] Borborygmus, diarrhea, abdominal distension, lumbar and hypochondriac pain.

A principle of herbal compatibility in the present disclosure is as follows.

In the formula, Radix Aconiti Lateralis Praeparata, being intensely acrid and hot in nature, enters the heart and kidney meridians. Combined with Ramulus Cinnamomi, which possesses a warm nature, the two herbs synergistically warm and tonify the heart and kidney, serving as the monarch drug (Jun) in the formula. Semen Sinapis Albae, Radix Kansui Praeparata, Semen Descurainiae function to resolve phlegm and promote diuresis, acting as the minister drugs (Chen). Eupolyphaga Steleophaga and Cortex Moutan invigorate blood circulation and unblock collaterals, serving as the assistant drugs (Zuo). Rhizoma Smilacis Glabrae, when used externally, drains dampness and relieves itching, acting as the guide drug (Shi) in the formula. HF is fundamentally characterized by deficiency, necessitating treatment strategies centered on the heart and kidneys. The formula integrates heart-kidney warming and tonifying herbs with blood-activating and meridian-dredging herbs, as well as dampness-resolving diuretics, administered through pattern-based differentiation and treatment. This approach simultaneously addresses both root deficiency and symptomatic manifestations by promoting blood circulation, unblocking meridians, inducing diuresis, and resolving dampness, thereby achieving dual Biao-Ben therapeutic efficacy and reliable clinical outcomes.

Additionally, this formula employs ginger juice, honey, and aged vinegar as excipients. An acupoint selection principle for the external application of the YANG-warming and heart-settling plaster focuses on warming and tonifying the heart and kidneys, promoting blood circulation, and dispelling water retention. Five acupoints are selected: Danzhong (CV17), Xinshu (BL15), Juque (CV14), Shenshu (BL23), and Jingmen (GB25). Among these, Xinshu (BL15) paired with Juque (CV14), and Shenshu (BL23) paired with Jingmen (GB25), form combinations for the heart and kidneys for the heart and kidneys, facilitating tonification of the heart and kidneys, promoting water drainage to resolve dampness, and activating blood circulation to unblock meridians. Danzhong (CV17), the front-mu point of the pericardium, serves as the convergence site for the Zong QI (ancestral QI) of the entire body. Its selection relies not only on its ability to tonify QI and unblock meridians to benefit the heart, but also on its capacity to activate YANG QI. The heart governs blood, while Danzhong governs QI; and QI generates and propels blood, and blood carries QI. These two aspects mutually reinforce each other.

In a preferred technical solution of the present disclosure, the TCM compound composition for treating HF, prepared from the following herbal raw materials in parts by weight:

2 parts of Semen Sinapis Albae, 2 parts of Radix Kansui Praeparata, 4 parts of Semen Descurainiae, 4 parts of Ramulus Cinnamomi, 3 parts of Radix Aconiti Lateralis Praeparata, 5 parts of Eupolyphaga Steleophaga, 5 parts of Cortex Moutan, and 3 parts of Rhizoma Smilacis Glabrae.

A preparation method of the YANG-warming and heart-settling plaster includes the steps of: pulverizing each herbal component in the prescribed dosage ratio into fine powder; mixing an appropriate amount of the compound composition with aged vinegar, ginger juice, and honey; and kneading this mixture into paste pills for external application via medicinal adhesive tape to the following acupoints: Danzhong (CV17), Xinshu (BL15), Juque (CV14), Shenshu (BL23), and Jingmen (GB25). Clinical verification shows that the present disclosure demonstrates its efficacy in treating HF, with favorable therapeutic effects.

The Chinese herbal compound composition provided in the present disclosure is composed of the following medicinal herbs: Semen Sinapis Albae, Radix Kansui Praeparata, Semen Lepidii, Ramulus Cinnamomi, Radix Aconiti Lateralis Praeparata, Eupolyphaga Steleophaga, Cortex Moutan, and Rhizoma Smilacis Glabrae. This formula is characterized by its dual approach, which involves simultaneous Biao-Ben treatment (addressing both symptoms and root causes) and concurrent heart-kidney regulation. It belongs to the category of distinctive TCM external application therapies, embodying the regional characteristics of Xin'an medicine. Particularly beneficial for improving HF symptoms (including chest stuffiness, palpitations, shortness of breath, cold intolerance, and edema) and related conditions, it demonstrates reliable therapeutic efficacy with an absence of toxic side effects.

### BRIEF DESCRIPTION OF THE DRAWINGS

FIG. 1 shows changes in brain natriuretic peptide (BNP) levels in HF patients before and after treatment with a TCM compound;

FIG. 2 shows changes in a 6-minute walk test (6MWT) in HF patients before and after treatment with a TCM compound; and

FIG. 3 shows changes in TCM syndrome scoring in HF patients before and after treatment with a TCM compound.

### DETAILED DESCRIPTION

The present disclosure is further described in detail in combination with the accompanying drawings and embodiments.

### Embodiment 1

### Preparation of YANG-Warming and Heart-Settling Plaster

Compound composition: 2 parts of Semen Sinapis Albae, 2 parts of Radix Kansui Praeparata, 4 parts of Semen Descurainiae, 4 parts of Ramulus Cinnamomi, 3 parts of Radix Aconiti Lateralis Praeparata, 5 parts of Eupolyphaga Steleophaga, 5 parts of Cortex Moutan, and 3 parts of Rhizoma Smilacis Glabrae.

Preparation method: each herbal component in the prescribed dosage ratio was pulverized into fine powder; an appropriate amount of the compound composition was mixed with 1 part of aged vinegar, 1 part of ginger juice, and 1 part of honey; and this mixture was kneaded into paste pills, which were divided into five equal portions for external application via medicinal adhesive tape: HuoXue Zhitong Gao (for non-allergic skin) or hypoallergenic surgical tape (for allergic skin). The pills were affixed to the following acupoints: Danzhong (CV17), Xinshu (BL15), Juque (CV14), Shenshu (BL23), and Jingmen (GB25). This process yielded the final product: YANG-warming and heart-settling plaster.

### Embodiment 2

The present disclosure enrolled 10 HF patients diagnosed at The First Affiliated Hospital of Anhui University of Chinese Medicine from 2023 to 2024 (patient demographics in Table 3). Participants received external application of the YANG-warming and heart-settling plaster (prepared according to Embodiment 1) at the following acupoints: Danzhong (CV17), Xinshu (BL15), Juque (CV14), Shenshu (BL23), and Jingmen (GB25). The plaster was administered once daily, with each session lasting 6-8 hours, and applied 5 times per week. Ten applications constituted one treatment course, spanning a total of two weeks. The study assessed changes in BNP levels, 6MWT, and TCM syndrome score before and after treatment with the formula to evaluate therapeutic efficacy.

1. BNP assisted in diagnosing HF and evaluating disease severity and prognosis.

All BNP measurements in this study were performed by the Clinical Biochemistry and Immunology Laboratory of The First Affiliated Hospital of Anhui University of Chinese Medicine using chemiluminescent immunoassay (CLIA) for BNP quantification. Specific steps were as follows.

(1) 0.5-1 mL of venous blood samples were collected from patients in Ethylenediaminetetraacetic acid (EDTA) vacuum tubes (non-fasting state permitted).

(2) BNP quantification was performed strictly adhering to the manufacturer's instructions for the BNP assay kit (chemiluminescent microparticle immunoassay, CMIA), utilizing instrument AHUCM/LAB-SOP-MY-YQ-013 under the operational framework of Abbott ARCHITECT i2000SR Automated Chemiluminescence Immunoassay System Standard Operating Procedure.

(3) Samples were mixed with paramagnetic particles coated with anti-BNP antibodies. BNP in the samples was bound to the paramagnetic particles coated with anti-BNP antibodies. Unbound components were subsequently removed through a washing procedure.

(4) Acridinium ester-labeled anti-BNP conjugate was added to form a reaction complex. Unbound conjugates were subsequently removed through a washing procedure.

(5) Pre-trigger solution and trigger solution were added, inducing acridinium ester chemiluminescence. The emitted light intensity was detected by the instrument. This light intensity exhibited a direct proportionality to the amount of acridinium ester conjugate, that is, the concentration of BNP, and was compared against a calibration curve stored in the instrument, which was derived from a series of standard solutions with known concentrations, thereby calculating the BNP concentration in the test sample.

2. 6MWT required patients to walk on a flat, hard surface at the fastest sustainable speed to measure the distance covered within six minutes. Specific steps were as follows.

(1) The principle of 6MWT was explained to the patients. Identical written instructions regarding the test protocol were provided to each participant. The patients were required to thoroughly review and sign a written informed consent document prior to participation.

(2) The patients needed to wear comfortable clothing and appropriate footwear during testing. Slippers, high-heeled shoes, or barefoot conditions are prohibited.

(3) A light meal was consumed prior to the test, with avoidance of heavy food intake immediately before the procedure.

(4) The patients were permitted to use daily walking aids during the test (such as canes or walkers).

(5) Strenuous exercise was avoided during the 2-hour period preceding the test. Warm-up activities were omitted. Similarly, vigorous physical exertion was prohibited for 2 hours following test completion.

(6) Prior to the test, the patients remained seated in chairs adjacent to the starting point for at least 15 minutes. During this period, potential contraindications were assessed. Test precautions were communicated to patients, including procedures for taking breaks and resuming the test. Demonstration of proper traffic cone navigation technique was provided as needed.

(7) A timer was adjusted (the timer was reset to zero and set to 6 minutes). All necessary equipment was positioned appropriately for test commencement.

(8) The patients were directed to position themselves at the starting line to initiate the test. During test execution, operators remained stationary near the starting line without accompanying the patient during walking. Throughout the test, standardized phrases of encouragement were delivered in a consistent tone and volume at predetermined intervals. Time updates were provided to the patients once per minute. A final warning was issued when 15 seconds remained. Upon test completion, the patients were instructed to remain stationary at their current positions. No additional encouraging language (or physical gestures) was permitted. If patients stopped walking before 6 minutes and declined to continue (or if operators determined continuation was contraindicated), the walking distance, stop time, and reason for premature termination were documented as specified in Table 1.

TABLE 1
6MWT measurement table

| | Start | End |
|---|---|---|
| Time | | |
| Heart rate | | |
| Dyspnea (Borg scale) | | |
| Fatigue (Borg scale) | | |
| SpO₂ (%) | | |
| Early Termination | Yes (reasons) | No |

| | |
|---|---|
| Termination Symptoms | Angina pectoris, dizziness, buttock |
| | pain, thigh pain, and calf pain |
| Actual Distance | |
| Predicted Distance | |
| Percentage of projected value | |
| Mild | 426-550 m |
| Moderate | 150-425 m |
| Severe | <150 m |

### 3. TCM Syndrome Score

This study employed a comparative assessment of TCM syndrome score before and after treatment to evaluate the formula's efficacy in ameliorating TCM syndrome manifestations, offering preliminary insights for clinical TCM management of HF.

This study was conducted by geriatric cardiologists from The First Affiliated Hospital of Anhui University of Chinese Medicine, following these specific procedures:

the clinical significance of the TCM syndrome score was explained to the patients. Identical protocol information was provided to all participants. After obtaining written informed consent, physicians systematically assessed each symptom criterion through patient interviews before and after HF treatment. Tongue coating and pulse diagnoses were performed concurrently. Scoring calculations were documented as detailed in Table 2.

TABLE 2
TCM syndrome score scale for HF

| | Normal | Mild | Moderate | Severe |
|---|---|---|---|---|
| Symptom | (0 scores) | (2 scores) | (4 scores) | (6 scores) |
| Palpitation | No symptoms | Mild palpitations | Distinct palpitations | Asymptomatic at |
| | | perceived during routine | during routine activities | rest, but palpitations |
| | | activities, without impact | that subside after rest, | immediately |
| | | on daily living or work | and daily activities can | provoked by |
| | | | be maintained with | minimal activity, |
| | | | difficulty | rendering daily |
| | | | | activities impossible |
| Shortness | No symptoms | Shortness of breath after | Shortness of breath after | Shortness of breath |
| of breath | | routine activities | minimal activity | even at rest without |
| | | | | activity |
| Fatigue and | No symptoms | Spiritless and fatigued, | Spiritless and | Severe fatigue, |
| lassitude | | but still able to persist | experiencing general | difficulty in |
| | | with routine work and | bodily fatigue, only | persisting with daily |
| | | daily life | barely able to persist | activities |
| | | | with daily activities | |
| Cough | No symptoms | Occasional cough with a | Frequent cough with a | Frequent cough |
| | | small amount of thin | moderate amount of thin | throughout the day |
| | | white sputum, and 24- | white sputum, and 24- | and night or |
| | | hour sputum volume 10- | hour sputum volume 50- | paroxysmal cough |
| | | 50 mL | 100 mL | with large amounts |
| | | | | of thin white or |
| | | | | frothy sputum, and |
| | | | | 24-hour sputum |
| | | | | volume over 100 mL |
| Chest | No symptoms | Occasionally experiences | Chest tightness (or pain) | Marked chest and |
| tightness | | chest pain or tightness | comes and goes | hypochondriac |
| | | | intermittently | tightness and pain |
| Limb | No symptoms | Mild facial edema upon | Edema of the lower | Generalized pitting |
| edema | | waking or pedal edema | limbs | edema (difficult to |
| | | | | rebound after |
| | | | | pressure) or |
| | | | | accompanied by |
| | | | | pleural |
| | | | | effusion/ascites |
| Cold | No symptoms | Cold hands and feet with | Cold limbs requiring | Whole-body |
| intolerance | | subjective chilliness | extra clothing/blankets | coldness not fully |
| | | | | relieved by added |
| | | | | clothing/blankets |
| Urine | No symptoms | 24 h urine volume above | 24 h urine volume 400- | 24 h urine volume |
| volume | | 1000 mL | 1000 mL | below 400 mL |
| Restlessness | Absent | Mental restlessness | Persistent agitation with | Manic behavior with |
| | | | inability to stay calm | loss of awareness |
| Perspiration | Dry, warm | Moist, warm skin | Clammy skin | Profuse whole-body |
| | skin | | | sweating drenching |
| | | | | clothes |
| Facial | Absent | 0 | Present | 2 scores |
| pallor | | | | |
| Bluish- | Absent | 0 | Present | 2 scores |
| purple lips | | | | |
| and mouth | | | | |
| Pale and | Absent | 0 | Present | 2 scores |
| dull tongue | | | | |
| Deep, thin, | Absent | 0 | Present | 2 scores |
| and | | | | |
| irregular | | | | |
| pulse | | | | |

### 4. Statistical Results

TABLE 3
Patient demographics and statistical results before and after treatment

| Serial | | BNP | | 6WMT | | Syndrome Score | |
|---|---|---|---|---|---|---|---|
| number | Name | Before | After | Before | After | Before | After |
| 1 | Zhu *C. | 338 | 97.2 | 300 | 450 | 34 | 22 |
| 2 | Tong *H. | 194 | 88.2 | 400 | 500 | 28 | 14 |
| 3 | Zhu *H. | 303.6 | 261.5 | 130 | 170 | 24 | 18 |
| 4 | Huang *Y. | 771 | 230.9 | 300 | 360 | 20 | 14 |
| 5 | Yu *Y. | 462 | 91.8 | 325 | 450 | 24 | 12 |
| 6 | Zhang *L. | 67 | 37.4 | 400 | 480 | 10 | 8 |
| 7 | Wang *R. | 247 | 95.9 | 325 | 410 | 18 | 10 |
| 8 | He *Q. | 932 | 395.7 | 200 | 350 | 28 | 18 |
| 9 | Chen *Z. | 125.4 | 117.9 | 250 | 310 | 18 | 16 |
| 10 | Xiao *X. | 505 | 383 | 270 | 300 | 24 | 22 |

| | | | | | | |
|---|---|---|---|---|---|---|
| Average value | 394.95 | 179.95 | 290 | 378 | 22.8 | 15.4 |

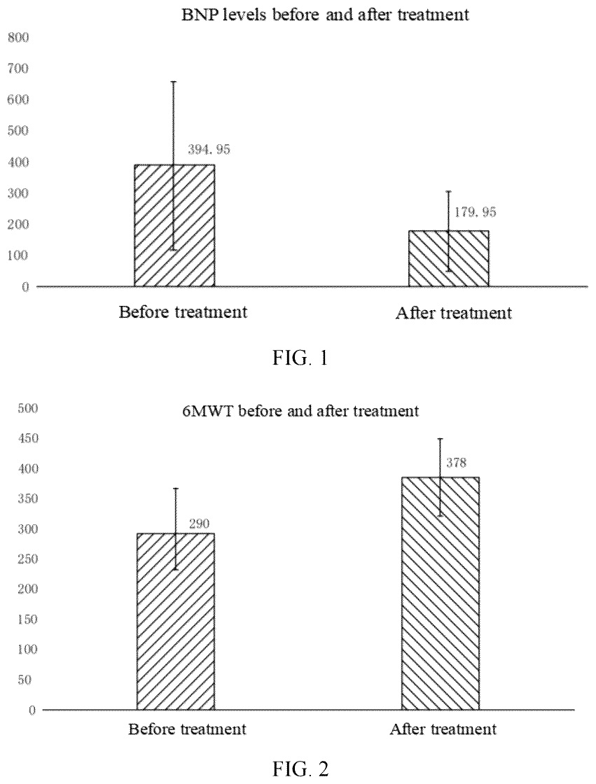

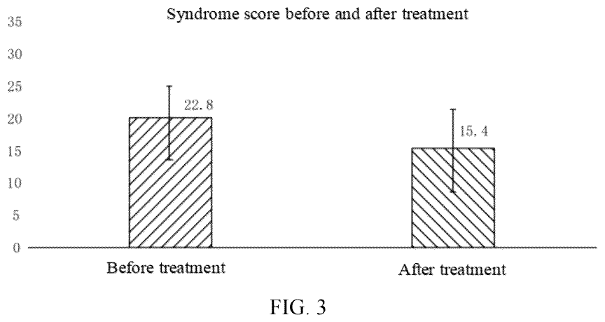

As shown in FIGS. 1-3 and Table 3, compared to baseline, post-treatment patients with HF demonstrate reduced BNP levels (P<0.05), significantly improved 6MWT performance (P<0.05), and decreased HF syndrome score (P<0.05). Consequently, the present disclosure provides an effective novel therapeutic agent for clinical management of HF, which is a refractory disease, holding substantial promise for broad clinical application.

The foregoing descriptions merely serve as examples and illustrations of the concepts of the present disclosure. A person skilled in the art may make various modifications, supplements, or substitutions with equivalent means to the specifically described embodiments without departing from the concept of the present disclosure or exceeding the scope defined by the claims, and these alterations fall within the scope of protection of the present disclosure.

1. A topical traditional Chinese medicine (TCM) compound composition for treating heart failure (HF), prepared from the following herbal raw materials in parts by weight:
1-3 parts of Semen Sinapis Albae, 1-3 parts of Radix Kansui Praeparata, 3-5 parts of Semen Descurainiae, 3-5 parts of Ramulus Cinnamomi, 2-4 parts of Radix Aconiti Lateralis Praeparata, 4-6 parts of Eupolyphaga Steleophaga, 4-6 parts of Cortex Moutan, and 2-4 parts of Rhizoma Smilacis Glabrae.

2. The topical TCM compound composition for treating HF according to claim 1, prepared from the following herbal raw materials in parts by weight:
2 parts of Semen Sinapis Albae, 2 parts of Radix Kansui Praeparata, 4 parts of Semen Descurainiae, 4 parts of Ramulus Cinnamomi, 3 parts of Radix Aconiti Lateralis Praeparata, 5 parts of Eupolyphaga Steleophaga, 5 parts of Cortex Moutan, and 3 parts of Rhizoma Smilacis Glabrae.

3. A YANG-warming and heart-settling plaster, prepared from the TCM compound composition according to claim 1, wherein a preparation method of the YANG-warming and heart-settling plaster comprises the steps of: pulverizing each herbal component in the prescribed dosage ratio into fine powder; mixing an appropriate amount of the compound composition with aged vinegar, ginger juice, and honey; and kneading this mixture into paste pills for external application via medicinal adhesive tape to the following acupoints: Danzhong (CV17), Xinshu (BL15), Juque (CV14), Shenshu (BL23), and Jingmen (GB25).

4. A YANG-warming and heart-settling plaster, prepared from the TCM compound composition according to claim 2, wherein a preparation method of the YANG-warming and heart-settling plaster comprises the steps of: pulverizing each herbal component in the prescribed dosage ratio into fine powder; mixing an appropriate amount of the compound composition with aged vinegar, ginger juice, and honey; and kneading this mixture into paste pills for external application via medicinal adhesive tape to the following acupoints: Danzhong (CV17), Xinshu (BL15), Juque (CV14), Shenshu (BL23), and Jingmen (GB25).
